(19) United States
(12) Patent Application Publication
(10) Pub. No.: US 2024/0141537 A1
(43) Pub. Date: May 2, 2024

(54) Superhydrophobic and Self-Cleaning Anticoagulant Composite Coating Material and Preparation Method and Use Thereof

(71) Applicant: Anhui Medical University, Hefei, AH (CN)

(72) Inventors: Shunli ZHENG, Hefei (CN); Qin RAO, Hefei (CN); Ling WENG, Hefei (CN); Jinshuang ZHANG, Hefei (CN); Donghao LIU, Hefei (CN); Quanli LI, Hefei (CN); Ying CAO, Hefei (CN); Jialong CHEN, Hefei (CN); Xiangyang LI, Hefei (CN); Hua QIU, Hefei (CN); Shengzhuo ZHANG, Hefei (CN); Daojun SHEN, Hefei (CN)

(73) Assignee: Anhui Medical University, Hefei, AH (CN)

(21) Appl. No.: 18/014,148

(22) PCT Filed: Nov. 8, 2022
(86) PCT No.: PCT/CN2022/130500

§ 371 Date: Dec. 31, 2022

(30) Foreign Application Priority Data
Oct. 31, 2022 (CN) 202211341328.0

(51) Int. Cl. C25D 11/26

(52) U.S. Cl. CPC C25D 11/26

(57) ABSTRACT

The present disclosure provides a superhydrophobic and self-cleaning anticoagulant composite coating material and a preparation method and use thereof, and relates to the technical field of biomedical materials. In the coating material provided by the present disclosure, a titanium dioxide nanotube-based structure increases microscopic roughness of a surface of a titanium-based metal substrate, and a hydrophobic modification layer reduces surface energy of the material. The rough structure and the hydrophobic modification layer have a synergistic effect to construct a superhydrophobic surface, making the surface of the material have self-cleaning characteristics and low adhesion. Air can be retained on the surface of the material to form an air layer, thereby reducing the contact area between the material and bacteria and platelets in the blood, and inhibiting adhesion of the bacteria, platelets, and plasma proteins to the material.

### CROSS REFERENCE TO RELATED APPLICATION

The present application claims priority to the Chinese Patent Application No. CN202211341328.0, filed with the China National Intellectual Property Administration (CNIPA) on Oct. 31, 2022, and entitled “SUPERHYDROPHOBIC AND SELF-CLEANING ANTICOAGULANT COMPOSITE COATING MATERIAL AND PREPARATION METHOD AND USE THEREOF”, which is incorporated herein by reference in its entirety.

### TECHNICAL FIELD

The present disclosure relates to the technical field of biomedical materials, in particular to a superhydrophobic and self-cleaning anticoagulant composite coating material and a preparation method and use thereof.

### BACKGROUND

Biomedical materials can be implanted into the human body and have excellent biocompatibility, repairing or replacing human diseased tissues or organs, or enhancing organ functions. With the aging of the global population and the improvement of people's quality of life, the demand for biomedical materials is increasing. Especially, the demand for biomedical materials continues to rise, including artificial joints, artificial teeth, and cardiovascular materials. However, this type of implant material faces two major complications, bacterial infection and thrombosis, which may lead to the implant to fail in the service process, resulting in physical and mental pains to the patients.

The bacterial infection and thrombus are mainly caused by colonization of microorganisms on the surface of implant materials and poor biocompatibility, and can be commonly treated through a combination of thrombolytic drugs and antibacterial agents clinically. However, excessive use of the thrombolytic drugs may trigger massive hemorrhage; meanwhile, long-term use of the antibacterial agents (such as antibiotics and antibacterial/bactericidal chemicals) may also lead to risks such as toxicity and drug resistance.

### SUMMARY

In view of this, an objective of the present disclosure is to provide a superhydrophobic and self-cleaning anticoagulant composite coating material and a preparation method and use thereof. In the present disclosure, the composite coating material has remarkable antibacterial and anticoagulant properties, and desirable biocompatibility, which can effectively inhibit the adhesion of bacteria and platelets.

To achieve the above objective of the present disclosure, the present disclosure provides the following technical solutions.

The present disclosure provides a superhydrophobic and self-cleaning anticoagulant composite coating material, including a titanium-based metal substrate, a titanium dioxide nanotube-based structure layer, and a hydrophobic modification layer that are sequentially laminated.

Preferably, the hydrophobic modification layer is formed by a hydrophobic modifier; and the hydrophobic modifier includes perfluorosilane and/or medium-chain and long-chain saturated fatty acids.

Preferably, the perfluorosilane includes perfluoroethoxysilane and/or perfluoromethoxysilane;
preferably, the perfluoroethoxysilane has a chemical formula of CF₃(CF₂)ₙCH₂CH₂Si(OC₂H₅)₃, and n is 5, 7, 9, or 11; and
preferably, the perfluoromethoxysilane has a chemical formula of CF₃(CF₂)ₘCH₂CH₂Si(OCH₃)₃, and m is 5 or 7.

Preferably, the medium-chain and long-chain saturated fatty acids are C₁₂ saturated fatty acids to C₂₂ saturated fatty acids.

Preferably, the C₁₂ saturated fatty acids to C₂₂ saturated fatty acids have a chemical formula of CH₃(CH₂)ₐCOOH, and a is 10 to 20.

The present disclosure further provides a preparation method of the superhydrophobic and self-cleaning anticoagulant composite coating material, including the following steps:
placing the titanium-based metal substrate into an electrolyte, conducting anodization to form a titanium dioxide nanotube-based structure on a surface of the titanium-based metal substrate, to obtain an anodized titanium dioxide coating material; and
immersing the anodized titanium dioxide coating material into a solution of the hydrophobic modifier to form a hydrophobic modification layer, to obtain the superhydrophobic and self-cleaning anticoagulant composite coating material.

Preferably, the electrolyte is selected from the group consisting of an aqueous inorganic salt alcoholic solution and an aqueous inorganic acid solution;
the aqueous inorganic salt alcoholic solution has 0.1 wt % to 0.5 wt % of an inorganic salt and 97.5 wt % to 97.9 wt % of an alcohol by concentration; and
the aqueous inorganic acid solution has a concentration of 0.1 wt % to 0.5 wt %.

Preferably, the inorganic salt in the aqueous inorganic salt alcoholic solution includes a fluoride salt and/or a sulfate salt.

Preferably, the fluoride salt is at least one selected from the group consisting of NH₄F, NaF and KF.

Preferably, the sulfate salt includes (NH₄)₂SO₄.

Preferably, the alcohol in the aqueous inorganic salt alcoholic solution is at least one selected from the group consisting of ethylene glycol, glycerol, and methanol.

Preferably, an inorganic acid in the aqueous inorganic acid solution includes hydrofluoric acid.

Preferably, the titanium-based metal substrate is selected from the group consisting of pure titanium, a titanium alloy sheet, and a titanium alloy foil.

Preferably, the titanium-based metal substrate is subjected to ultrasonic alcohol washing and ultrasonic water washing in sequence before use.

Preferably, the ultrasonic alcohol washing and the ultrasonic water washing each are conducted at 20° C. to 30° C. for 2 min to 10 min.

Preferably, a cathode used in the anodization is prepared by a material selected from the group consisting of graphite, a platinum sheet, and a stainless steel.

Preferably, during the anodization, an anode and the cathode have a spacing distance of 2 cm to 8 cm.

Preferably, the anodization is conducted at 0° C. to 30° C. with a voltage of 15 V to 55 V for 0.5 h to 5 h.

Preferably, the solution of the hydrophobic modifier has a concentration of 0.5 wt % to 10 wt %.

Preferably, the solution of the hydrophobic modifier has a solvent of alcohol.

Preferably, the immersing is conducted for 0.5 h to 24 h.

Preferably, the preparation method further includes washing and drying an immersed material; where the drying is conducted at 80° C. to 140° C. for 0.5 h to 24 h.

The present disclosure further provides use of the superhydrophobic and self-cleaning anticoagulant composite coating material or a superhydrophobic and self-cleaning anticoagulant composite coating material prepared by the preparation method in preparation of a bacteriostatic and anticoagulant biomedical material.

Preferably, the superhydrophobic and self-cleaning anticoagulant composite coating material is applied as a surface coating of a cardiovascular stent or a surface coating of an implant.

The present disclosure provides a superhydrophobic and self-cleaning anticoagulant composite coating material, including a titanium-based metal substrate, a titanium dioxide nanotube-based structure layer, and a hydrophobic modification layer that are sequentially laminated. In the superhydrophobic and self-cleaning anticoagulant composite coating material provided by the present disclosure, a titanium dioxide nanotube-based structure increases microscopic roughness of a surface of a titanium-based metal substrate, and a hydrophobic modification layer reduces surface energy of the material. The rough structure and the hydrophobic modification layer have a synergistic effect to construct a superhydrophobic surface, making the surface of the material have self-cleaning characteristics and low adhesion. Air can be retained on the surface of the material to form an air layer, thereby reducing a contact area between the material and bacteria and platelets in the blood, and inhibiting adhesion of the bacteria, platelets, and plasma proteins to the material. Therefore, the material has an excellent self-cleaning performance of “anti-biofouling”, anticoagulant properties, and desirable biocompatibility, showing a satisfactory application prospect as a biomedical material.

The present disclosure further provides a preparation method of the superhydrophobic and self-cleaning anticoagulant composite coating material. In the present disclosure, the preparation method has simple operations, a low production cost, and environmental friendliness, which is suitable for industrial production.

### BRIEF DESCRIPTION OF THE DRAWINGS

FIG. 1A-C show scanning electron microscopy (SEM) images of a titanium-based coating material prepared in Example 5 and a clean bare titanium foil prepared in Comparative Example 1; where (a) is a front shape of Comparative Example 1, (b) is a front shape of Example 5, and (c) is a cross-sectional shape of Example 5;

FIG. 2 shows contact angle values on surfaces of titanium-based coating materials prepared in Example 5, Comparative Example 6, and Comparative Example 11, and the clean bare titanium foil prepared in Comparative Example 1; where SH-35 V is Example 5, Ti is Comparative Example 1, AO-35 V is Comparative Example 6, and Ti+FAS is Comparative Example 11;

FIG. 3 shows cell viability diagram of co-culture on extracts of the titanium-based coating materials prepared in Example 5, Comparative Example 6, and Comparative Example 11, and the clean bare titanium foil prepared in Comparative Example 1 with endothelial cells for 1 d, 3 d, and 5 d; where SH-35 V is Example 5, Ti is Comparative Example 1, AO-35 V is Comparative Example 6, and Ti+FAS is Comparative Example 11;

FIG. 4 shows cytocompatibility fluorescence microscope images of the extracts of the titanium-based coating materials prepared in Example 5, Comparative Example 6, and Comparative Example 11, and the clean bare titanium foil prepared in Comparative Example 1, and a blank control group; where (a₁) to (a₃) are the blank control group, (b₁) to (b₃) are Comparative Example 1, (c₁) to (c₃) are Comparative Example 6, (d₁) to (d₃) are Comparative Example 11, and (e₁) to (e₃) are Example 5;

FIG. 5A-D show bacterial adhesion on surfaces of the titanium-based coating materials prepared in Example 5, Comparative Example 6, and Comparative Example 11, and the clean bare titanium foil prepared in Comparative Example 1; where FIG. 5A is Comparative Example 1, FIG. 5B is Comparative Example 6, FIG. 5C is Comparative Example 11, and FIG. 5D is Example 5;

FIG. 6 shows hemolysis rates of the titanium-based coating materials prepared in Example 5, Comparative Example 6, and Comparative Example 11, and the clean bare titanium foil prepared in Comparative Example 1; where SH-35 V is Example 5, Ti is Comparative Example 1, AO-35 V is Comparative Example 6, and Ti+FAS is Comparative Example 11;

FIG. 7 shows SEM images of static adhesion with platelets on the surfaces of the titanium-based coating materials prepared in Example 5, Comparative Example 6, and Comparative Example 11, and the clean bare titanium foil prepared in Comparative Example 1; where (a₁) to (a₂) are Comparative Example 1, (b₁) to (b₂) are Comparative Example 6, (c₁) to (c₂) are Comparative Example 11, and (d₁) to (d₂) are Example 5; and

FIG. 8 shows SEM images of dynamic adhesion with platelets on the surfaces of the titanium-based coating material prepared in Example 5 and the clean bare titanium foil prepared in Comparative Example 1; where (a₁) to (a₂) are Comparative Example 1, and (b₁) to (b₂) are Example 5.

### DETAILED DESCRIPTION OF THE EMBODIMENTS

The present disclosure provides a superhydrophobic and self-cleaning anticoagulant composite coating material, including a titanium-based metal substrate, a titanium dioxide nanotube-based structure layer, and a hydrophobic modification layer that are sequentially laminated.

In the present disclosure, the hydrophobic modification layer is formed by a hydrophobic modifier; and the hydrophobic modifier includes preferably perfluorosilane and/or medium-chain and long-chain saturated fatty acids. The perfluorosilane includes preferably perfluoroethoxysilane and/or perfluoromethoxysilane; the perfluoroethoxysilane has a chemical formula of preferably CF₃(CF₂)ₙCH₂CH₂Si(OC₂H₅)₃, and n is preferably 5, 7, 9, or 11; specifically, the perfluoroethoxysilane is preferably at least one selected from the group consisting of 1H,1H,2H,2H-perfluorodecyltriethoxysilane (CF₃(CF₂)₇CH₂CH₂Si(OC₂H₅)₃), 1H,1H,2H,2H-perfluorododecyltriethoxysilane (CF₃(CF₂)₉CH₂CH₂Si(OC₂H₅)₃), and 1H,1H,2H,2H-perfluorotetradecyltriethoxysilane (CF₃(CF₂)₁₁CH₂CH₂Si(OC₂H₅)₃); and the perfluoromethoxysilane has a chemical formula of preferably CF₃(CF₂)ₘCH₂CH₂Si(OCH₃)₃, and m is preferably 5 or 7. The medium-chain and long-chain saturated fatty acids are preferably C₁₂ saturated fatty acids to C₂₂ saturated fatty acids; the C₁₂ saturated fatty acids to C₂₂ saturated fatty acids have a chemical formula of CH₃(CH₂)ₐCOOH, and a is 10 to 20, preferably 10, 11, 12, 13, 14, 15, 16, 17, 18, 19, or 20.

The present disclosure further provides a preparation method of the superhydrophobic and self-cleaning anticoagulant composite coating material, including the following steps:
placing the titanium-based metal substrate into an electrolyte, conducting anodization to form a titanium dioxide nanotube-based structure on a surface of the titanium-based metal substrate, to obtain an anodized titanium dioxide coating material; and
immersing the anodized titanium dioxide coating material into a solution of the hydrophobic modifier to form a hydrophobic modification layer, to obtain the superhydrophobic and self-cleaning anticoagulant composite coating material.

In the present disclosure, unless otherwise specified, all raw material components are commercially available products well known to persons skilled in the art.

In the present disclosure, the titanium-based metal substrate is placed into an electrolyte, anodization is conducted to form a titanium dioxide nanotube-based structure on a surface of the titanium-based metal substrate, to obtain an anodized titanium dioxide coating material.

In the present disclosure, the titanium-based metal substrate (as an anode) is preferably selected from the group consisting of pure titanium, a titanium alloy sheet, and a titanium alloy foil. In the present disclosure, the titanium-based metal substrate is preferably cleaned before use, and the cleaning is conducted by preferably ultrasonic alcohol washing and ultrasonic water washing in sequence; the ultrasonic alcohol washing and the ultrasonic water washing each are conducted at preferably 20° C. to 30° C., more preferably 25° C. for preferably 2 min to 10 min, more preferably 5 min; the ultrasonic alcohol washing is conducted by preferably absolute ethanol; the ultrasonic water washing is conducted by preferably deionized water.

In the present disclosure, the electrolyte is preferably selected from the group consisting of an aqueous inorganic salt alcoholic solution and an aqueous inorganic acid solution. The inorganic salt in the aqueous inorganic salt alcoholic solution includes preferably a fluoride salt and/or a sulfate salt; the fluoride salt is preferably at least one selected from the group consisting of NH₄F, NaF and KF; the sulfate salt includes preferably (NH₄)₂SO₄; the alcohol in the aqueous inorganic salt alcoholic solution is preferably at least one selected from the group consisting of ethylene glycol, glycerol, and methanol, more preferably includes the ethylene glycol, the glycerol, or the methanol, and even more preferably the ethylene glycol. The aqueous inorganic salt alcoholic solution has preferably 0.1 wt % to 0.5 wt %, more preferably 0.15 wt % to 0.45 wt %, and even more preferably 0.25 wt % of the inorganic salt; and the aqueous inorganic salt alcoholic solution has preferably 97.5 wt % to 97.9 wt %, more preferably 97.55 wt % to 97.85 wt %, and even more preferably 97.75 wt % of the alcohol by concentration. The inorganic acid in the aqueous inorganic acid solution includes preferably hydrofluoric acid; and the aqueous inorganic acid solution has preferably 0.1 wt % to 0.5 wt %, more preferably 0.2 wt % to 0.3 wt % of the inorganic acid.

In the present disclosure, a preparation method of the aqueous inorganic salt alcoholic solution includes preferably the following steps: mixing the inorganic salt with the alcohol, and then mixing with water. The formation of titanium dioxide nanotubes is greatly affected by dissociation of the electrolyte. If the electrolyte dissociates too quickly, it is not easy to obtain an ideal nanotube array, and the inorganic salt is not easy to dissociate quickly in the alcohol. Therefore, adding the inorganic salt to the alcohol and then adding water is beneficial to the formation of titanium dioxide nanotubes.

In the present disclosure, a cathode used in the anodization is prepared by a material preferably selected from the group consisting of graphite, a platinum sheet, and a stainless steel.

In the present disclosure, the anodization is conducted at preferably 0° C. to 30° C., more preferably 10° C. to 25° C., even more preferably at a room temperature (25° C.) in an example with a voltage of preferably 15 V to 55 V, more preferably 15 V to 40 V, and even more preferably 30 V to 35 V for preferably 0.5 h to 5 h, more preferably 0.5 h to 3 h, and even more preferably 1 h to 2 h; during the anodization, the anode and the cathode have a spacing distance of preferably 2 cm to 8 cm, more preferably 3 cm to 6 cm, and even more preferably 4 cm to 5 cm.

In the present disclosure, the anodized titanium dioxide coating material is immersed into a solution of the hydrophobic modifier to form a hydrophobic modification layer, to obtain the superhydrophobic and self-cleaning anticoagulant composite coating material.

In the present disclosure, the solution of the hydrophobic modifier has a concentration of preferably 0.5 wt % to 10 wt %, more preferably 1 wt % to 10 wt %, and even more preferably 1 wt % to 5 wt %; and the solution of the hydrophobic modifier has a solvent of preferably alcohol, including preferably ethanol and/or methanol.

In the present disclosure, the immersing is conducted at preferably a room temperature for preferably 0.5 h to 24 h, more preferably 0.5 h to 5 h, and even more preferably 0.5 h to 1 h.

In the present disclosure, a titanium dioxide nanotube-based coating prepared by the anodization is hydrophilic and has hydroxyl (—OH) groups on a surface. For perfluorosilane, due to the presence of hydrophobic groups —CF₂ and —CF₃, the perfluorosilane has highly low surface energy, so as to effectively reduce the surface energy of the sample. The mechanism of action includes: one end of the molecule is a polar functional group —Si(OC₂H₅)₃ or —Si(OCH₃)₃, and the other end is composed of a long hydrophobic chain; the functional group —Si(OC₂H₅)₃ or —Si(OCH₃)₃ are hydrolyzed to form silanol (Si—OH), which serves as a highly-active reaction intermediate; the silane molecule undergoes dehydration condensation with —OH on the titanium substrate through Si—OH, forming a self-assembled monolayer with low surface energy on the surface of the titanium-based material. Meanwhile, the intermolecular Si—OH produces vertical polymerization through dehydration to form graft polysiloxane, increasing hydrophobicity of the coating surface. For medium- and long-chain saturated fatty acids, the mechanism of action includes: an alkyl hydrophobic long carbon chain at one end is grafted to the titanium-based surface by forming carboxylates, reducing the surface energy and increasing the hydrophobicity of the coating surface.

In the present disclosure, the preparation method further includes washing and drying an immersed material to obtain the superhydrophobic and self-cleaning anticoagulant composite coating material. The drying is conducted at preferably 80° C. to 140° C., more preferably 100° C. to 120° C. for preferably 0.5 h to 24 h, more preferably 1 h to 2 h.

The present disclosure further provides use of the superhydrophobic and self-cleaning anticoagulant composite coating material or a superhydrophobic and self-cleaning anticoagulant composite coating material prepared by the preparation method in preparation of a bacteriostatic and anticoagulant biomedical material. In the present disclosure, the superhydrophobic and self-cleaning anticoagulant composite coating material is preferably applied as a surface coating of a cardiovascular stent or a surface coating of an implant. By functionalizing an implant material (the titanium-based metal substrate) itself, a self-cleaning coating material with desirable biocompatibility is constructed on the implant surface, without protein, bacteria, or plasma adhesion, to solve bacterial infection and thrombus from the perspective of source etiology.

Specifically, the titanium dioxide nanotubes in the superhydrophobic and self-cleaning anticoagulant composite coating material have a rough structure, and a synergistic effect of the rough structure with the hydrophobic modification layer (low-surface-energy substance modification) forms a superhydrophobic surface, making the composite coating material have an excellent self-cleaning performance of “anti-biofouling”, anticoagulant properties, and desirable biocompatibility. The coating can be applied to biomedical materials in contact with human blood and tissues, especially as a surface coating of cardiovascular stents or implants, showing desirable application prospects.

The technical solutions of the present disclosure will be described below clearly and completely in conjunction with the examples of the present disclosure. Apparently, the described examples are only a part of, not all of, the examples of the present disclosure. All other embodiments obtained by those of ordinary skill in the art based on the embodiments of the present disclosure without creative efforts shall fall within the protection scope of the present disclosure.

In the following examples and comparative examples:

An electrolyte is 0.25 wt % NH₄F-97.75 wt % ethylene glycol-2 wt % aqueous solution, which is ready-to-use and ready-made. A preparation method thereof is as follows: mixing the NH₄F with the ethylene glycol well, and then mixing with water well.

A titanium foil is ultrasonically washed with absolute ethanol for 5 min, then with deionized water for 5 min, and dried under an inert gas (argon or nitrogen) to obtain a clean bare titanium foil.

### Example 1

(1) By a method of anodization, the anodization was conducted with a clean bare titanium foil as an anode and a graphite plate as a cathode, in an electrolytic cell filled with 0.25 wt % NH₄F-97.75 wt % ethylene glycol-2 wt % aqueous solution, under a spacing distance of 4 cm between the cathode and the anode, at a room temperature and 15 V for 1 h, and a titanium dioxide nanotube-based structure (denoted as AO-15 V) was formed on a surface of the titanium foil, to obtain an anodized titanium dioxide coating material.

(2) The anodized titanium dioxide coating material was placed in a 1 wt % ethanol solution of 1H,1H,2H,2H-perfluorodecyltriethoxysilane (FAS) and immersed for 30 min at a room temperature, washed with deionized water, and then dried in an oven at 100° C. for 1 h to obtain a superhydrophobic and self-cleaning anticoagulant composite coating material (denoted as SH-15 V).

### Examples 2 to 9

The superhydrophobic and self-cleaning anticoagulant composite coating materials were prepared according to the method of Example 1, where preparation conditions of Examples 2 to 9 were shown in Table 1.

TABLE 1
Preparation conditions of Examples 1 to 9

| | Anodization | Anodization | FAS modification | Heat- |
|---|---|---|---|---|
| | (AO) voltage | (AO) time | time | drying time |
| Example 1 | 15 V | 1 h | 0.5 h | 1 h |
| Example 2 | 20 V | 1 h | 0.5 h | 1 h |
| Example 3 | 25 V | 1 h | 0.5 h | 1 h |
| Example 4 | 30 V | 1 h | 0.5 h | 1 h |
| Example 5 | 35 V | 1 h | 0.5 h | 1 h |
| Example 6 | 40 V | 1 h | 0.5 h | 1 h |
| Example 7 | 45 V | 1 h | 0.5 h | 1 h |
| Example 8 | 50 V | 1 h | 0.5 h | 1 h |
| Example 9 | 55 V | 1 h | 0.5 h | 1 h |

### Comparative Example 1

A clean bare titanium foil (Ti) was used.

### Comparative Example 2

An anodized titanium dioxide coating material (denoted as AO-15 V) was prepared according to the method of Example 1.

### Comparative Examples 3 to 10

The anodized titanium dioxide coating materials were prepared according to the method of Comparative Example 2, where preparation conditions of Comparative Examples 3 to 10 were shown in Table 2.

TABLE 2
Preparation conditions of Comparative Examples 2 to 10

| | Anodization (AO) | Anodization (AO) |
|---|---|---|
| | voltage | time |
| Comparative Example 2 | 15 V | 1 h |
| Comparative Example 3 | 20 V | 1 h |
| Comparative Example 4 | 25 V | 1 h |
| Comparative Example 5 | 30 V | 1 h |
| Comparative Example 6 | 35 V | 1 h |
| Comparative Example 7 | 40 V | 1 h |
| Comparative Example 8 | 45 V | 1 h |
| Comparative Example 9 | 50 V | 1 h |
| Comparative Example 10 | 55 V | 1 h |

### Comparative Example 11

The clean titanium foil was placed in a FAS ethanol solution with a concentration of 1 wt %, immersed at a room temperature for 30 min, washed with deionized water, and dried in an oven at 100° C. for 1 h to obtain a hydrophobic titanium-based coating material (denoted as Ti+FAS).

### Test Example

Taking Example 5 (SH-35 V) as an example, the surface morphology and performances are studied for the superhydrophobic and self-cleaning anticoagulant composite coating material prepared by the present disclosure.

(1) Microscopic Morphology

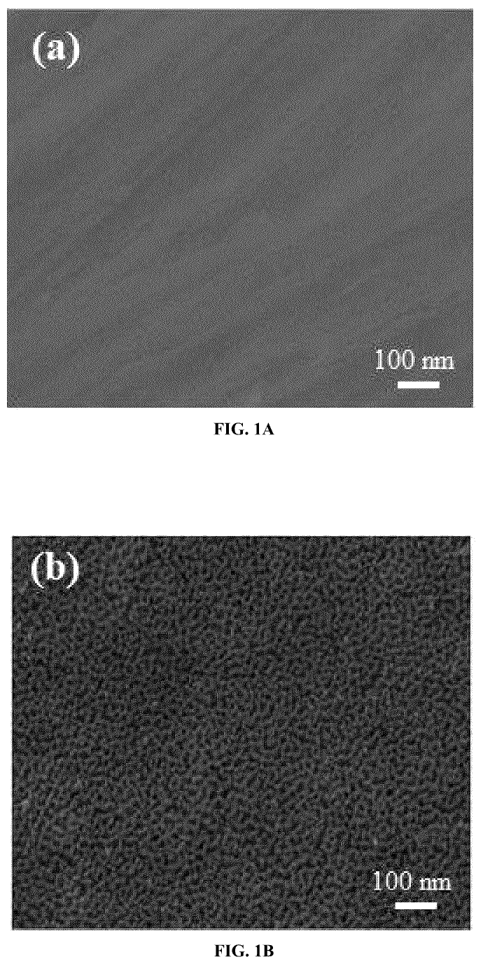

FIG. 1A-C showed SEM images of the titanium-based coating material prepared in Example 5 and the clean bare titanium foil prepared in Comparative Example 1; where FIG. 1A was a front shape of Comparative Example 1, FIG. 1B was a front shape of Example 5, and FIG. 1C was a cross-sectional shape of Example 5. It was seen from FIG. 1A that the surface of the titanium foil had a relatively smooth appearance; the surface of the superhydrophobic and self-cleaning anticoagulant composite coating material presented a rough nano-porous structure of titanium dioxide (TiO₂) (FIG. 1B)); the cross-section can show a titanium dioxide (TiO₂) nanotube structure (FIG. 1C). Since the construction of a microstructure of the sample surface is controlled by anodization, the modifier FAS could form a self-assembled monolayer with low surface energy on the surface of the material, which did not affect the microstructure.

(2) Contact Angle of Material Surface

Test method: 5 μL of water droplets were placed on surfaces of the titanium-based coating materials prepared in Example 5, Comparative Example 6, and Comparative Example 11 and the clean bare titanium foil prepared in Comparative Example 1, and the contact angle value was measured on the surface of the titanium-based coating materials.

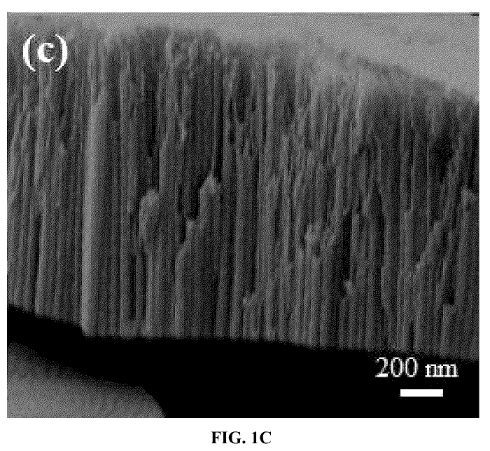

FIG. 2 showed contact angle values on surfaces of the titanium-based coating materials prepared in Example 5, Comparative Example 6, and Comparative Example 11, and the clean bare titanium foil prepared in Comparative Example 1. It was seen from FIG. 2 that the clean bare titanium foil (Ti) prepared in Comparative Example 1, the anodized titanium dioxide coating material (AO-35 V) prepared in Comparative Example 6 at a voltage of 35 V, and the hydrophobic titanium-based coating material (Ti+FAS) prepared in Comparative Example 11 had contact angles of 82.2±4.5°, 8.9±1.2°, and 104.2±10.2° on the surfaces, respectively. The superhydrophobic and self-cleaning anticoagulant composite coating material prepared in Example 5 had a contact angle of 164.9±2.8° on the surface, showing a superhydrophobic state. The surface of the superhydrophobic and self-cleaning anticoagulant composite coating material could retain air to form an air layer, which can reduce the contact area between the surface of the material and water, thus reducing the adhesion with water to play the role of hydrophobic self-cleaning effect.

(3) Biocompatibility

Test method: the superhydrophobic and self-cleaning anticoagulant composite coating material prepared in Example 5, the clean bare titanium foil prepared in Comparative Example 1, the anodized titanium dioxide coating material prepared in Comparative Example 6, and the hydrophobic titanium-based coating material prepared in Comparative Example 11 were placed in 90 wt % DMEM/F12+10 wt % fetal bovine serum separately, and extracted for 3 d to obtain an extract to be tested for each group. The 90 wt % DMEM/F12+10 wt % fetal bovine serum was used as a blank control.

By an extraction solution method, endothelial cells (EC) (1×10⁴ cells/cm²) were separately co-cultured with the extracts to be tested for 1 d, 3 d, and 5 d, and then tested by a Cell Counting Kit-8 (CCK-8) kit to determine the number of live cells; after rhodamine 123 staining, the growth state and cell proliferation of the cells were observed with a fluorescence microscope.

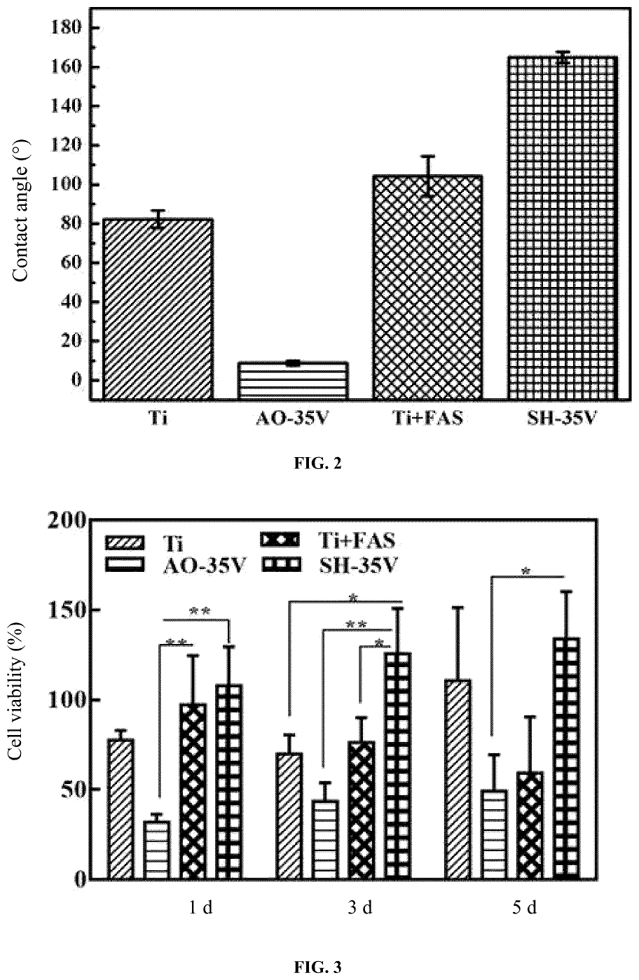

FIG. 3 showed the cell viability of Example 5, Comparative Example 1, Comparative Example 6, and Comparative Example 11 co-cultured with endothelial cells for 1 d, 3 d, and 5 d. It was seen from the figure that compared with the extracts of the clean bare titanium foil (Comparative Example 1), the anodized titanium dioxide coating material (Comparative Example 6), and the hydrophobic titanium-based coating material (Comparative Example 11), the extract of the superhydrophobic and self-cleaning anticoagulant composite coating material prepared in Example 5 could obviously observe more surviving cells; and with the increase of test time, the number of viable cells increased significantly.

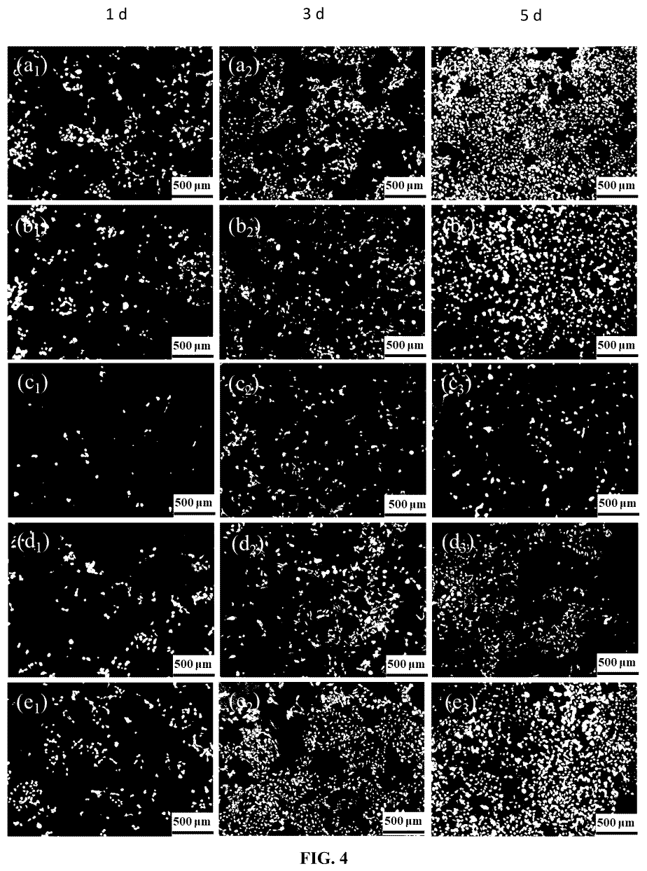

FIG. 4 showed cytocompatibility fluorescence microscope images of the superhydrophobic and self-cleaning anticoagulant composite coating material prepared in Example 5, the clean bare titanium foil prepared in Comparative Example 1, the anodized titanium dioxide coating material prepared in Comparative Example 6, and the hydrophobic titanium-based coating material prepared in Comparative Example 11; where (a₁) to (a₃) were the blank control group, (b₁) to (b₃) were Comparative Example 1, (c₁) to (c₃) were Comparative Example 6, (d₁) to (d₃) were Comparative Example 11, and (e₁) to (e₃) were Example 5. Through (e₁) to (e₃), it was seen that the experimental group co-cultured with the extract of the superhydrophobic and self-cleaning anticoagulant composite coating material prepared in Example 5 had a high cell proliferation density and desirable cell morphology, which was better than those of the clean bare titanium foil group (Comparative Example 1), and was much better than those of the anodized titanium dioxide coating material group (Comparative Example 6), and the hydrophobic titanium-based coating material group (Comparative Example 11).

The above experiments showed that, the superhydrophobic and self-cleaning anticoagulant composite coating material had more desirable biocompatibility compared with the clean bare titanium foil, anodized titanium dioxide coating material, and hydrophobic titanium-based coating material.

(4) Bacterial Adhesion on the Surface

Test method: the superhydrophobic and self-cleaning anticoagulant composite coating material prepared in Example 5, the clean bare titanium foil prepared in Comparative Example 1, the anodized titanium dioxide coating material prepared in Comparative Example 6, and the hydrophobic titanium-based coating material prepared in Comparative Example 11 were co-cultured with Staphylococcus aureus for 12 h, diluted 10³ times after sampling, rinsing, and ultrasonication, and then coated on a plate to observe the bacterial adhesion on the surface of the sample.

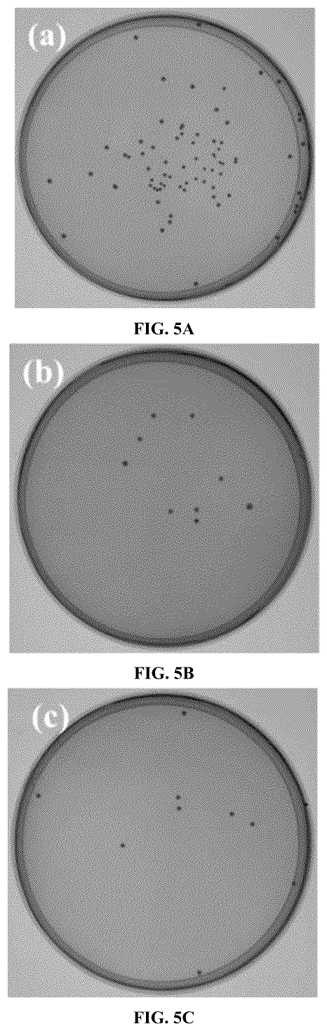

FIG. 5A-D showed bacterial adhesion on surfaces of the superhydrophobic and self-cleaning anticoagulant composite coating material prepared in Example 5, the clean bare titanium foil prepared in Comparative Example 1, the anodized titanium dioxide coating material prepared in Comparative Example 6, and the hydrophobic titanium-based coating material prepared in Comparative Example 11; where FIG. 5A was Comparative Example 1, FIG. 5B was Comparative Example 6, FIG. 5C was Comparative Example 11, and FIG. 5D was Example 5. It was seen from FIG. 5D that the surface of the superhydrophobic and self-cleaning anticoagulant composite coating material prepared in Example 5 had only an extremely small amount of bacterial adhesion, proving that the coating material could effectively inhibit bacterial adhesion, showing a desirable “anti-biofouling” performance. However, the surfaces of the clean bare titanium foil group (Comparative Example 1), the anodized titanium dioxide coating material group (Comparative Example 6), and the hydrophobic titanium-based coating material group (Comparative Example 11) each were adhered with a certain amount of S. aureus to varying degrees. This showed that the superhydrophobic and self-cleaning anticoagulant composite coating material could effectively inhibit the adhesion of bacteria and had an excellent self-cleaning performance of “anti-biofouling” compared with the clean bare titanium foil, anodized titanium dioxide coating material and hydrophobic titanium-based coating material.

(5) Blood Compatibility Test

(5.1) Hemolytic Performance Test

Test method: fresh anticoagulated rabbit blood was diluted with a 0.9 wt % NaCl solution (normal saline) at a volume ratio of 4:5. The superhydrophobic and self-cleaning anticoagulant composite coating material prepared in Example 5, the clean bare titanium foil prepared in Comparative Example 1, the anodized titanium dioxide coating material prepared in Comparative Example 6, and the hydrophobic titanium-based coating material prepared in Comparative Example 11 were placed in 9.8 mL of the normal saline, kept in a water bath at 37° C. for 30 min, added with 0.2 mL of diluted blood, shaken gently, and kept warm in water for 60 min. The sample was centrifuged at 3,000 r/min for 5 min, a supernatant was transferred to a clean 96-well plate, and an absorbance value was measured at a wavelength of 540 nm as an indicator of hemolysis. A negative control was 9.8 mL of normal saline+0.2 mL of diluted blood, and a positive control was 9.8 mL of distilled water+0.2 mL of diluted blood.

A calculation formula of hemolysis rate was as follows: hemolysis rate=(Dₜ−D_{nc})/(D_{pc}−D_{nc})×100%; where

Dₜ was an absorbance of the sample (a greater Dₜ meant a greater hemolysis rate), D_{nc} was an absorbance of the negative control, and D_{pc} was an absorbance of the positive control. In the hemolysis experiment, the hemolysis rate of the negative control group was 0, and the hemolysis rate of the positive control group was 100%. According to national standards, a hemolysis rate exceeding 5% indicated that the test material had hemolysis, and a hemolysis rate lower than 5% indicated that it met the requirements of hemolysis test for biomedical materials.

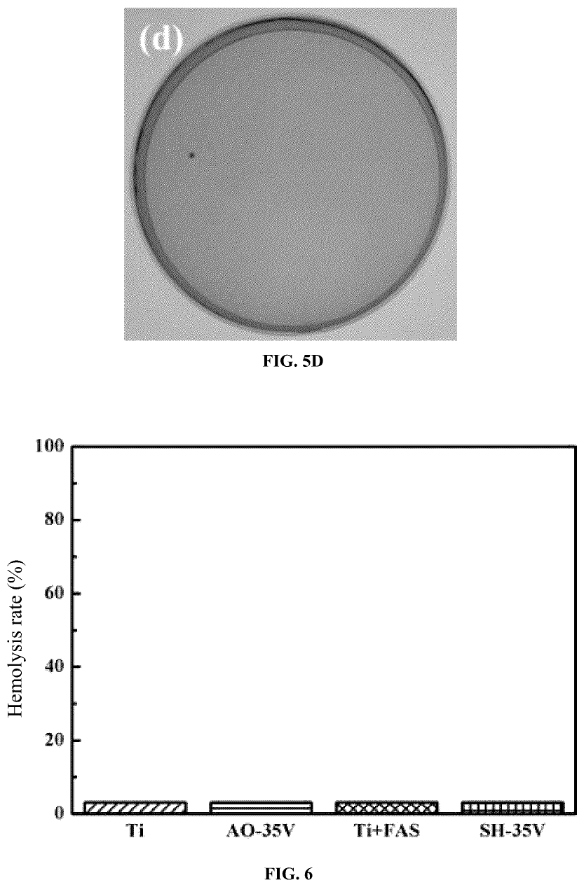

FIG. 6 shows hemolysis rates of the titanium-based coating materials prepared in Example 5, Comparative Example 6, and Comparative Example 11, and the clean bare titanium foil prepared in Comparative Example 1; where SH-35 V was Example 5, Ti was Comparative Example 1, AO-35 V was Comparative Example 6, and Ti+FAS was Comparative Example 11. As shown in the figure, the titanium-based coating materials prepared by Example 5 and Comparative Examples 6 and 11 had the same hemolysis rate as that of the clean bare titanium foil of Comparative Example 1, which was lower than 5%, indicating that the coating material had no hemolysis effect and met the requirements of the hemolysis test for biomedical materials.

### (5.2) Static Anticoagulant Performance—Static Platelet Adhesion Test In Vitro

Test method: fresh blood from the ear vein of live rabbits was centrifuged at 1,500 r/min for 15 min, and a platelet-rich plasma (PRP) in the upper layer was absorbed and placed in a 12-well plate. The superhydrophobic and self-cleaning anticoagulant composite coating material prepared in Example 5, the clean bare titanium foil prepared in Comparative Example 1, the anodized titanium dioxide coating material prepared in Comparative Example 6, and the hydrophobic titanium-based coating material prepared in Comparative Example 11 were immersed in 500 μL of the platelet-rich plasma per well, incubated in a constant-temperature water bath at 37° C. for 45 min, and gently washed with a PBS solution 3 times until the unadhered platelets on the samples were washed away. After fixation, dehydration, critical point drying, and gold spraying, the adhesion of platelets on the surface of the samples was observed with a SEM.

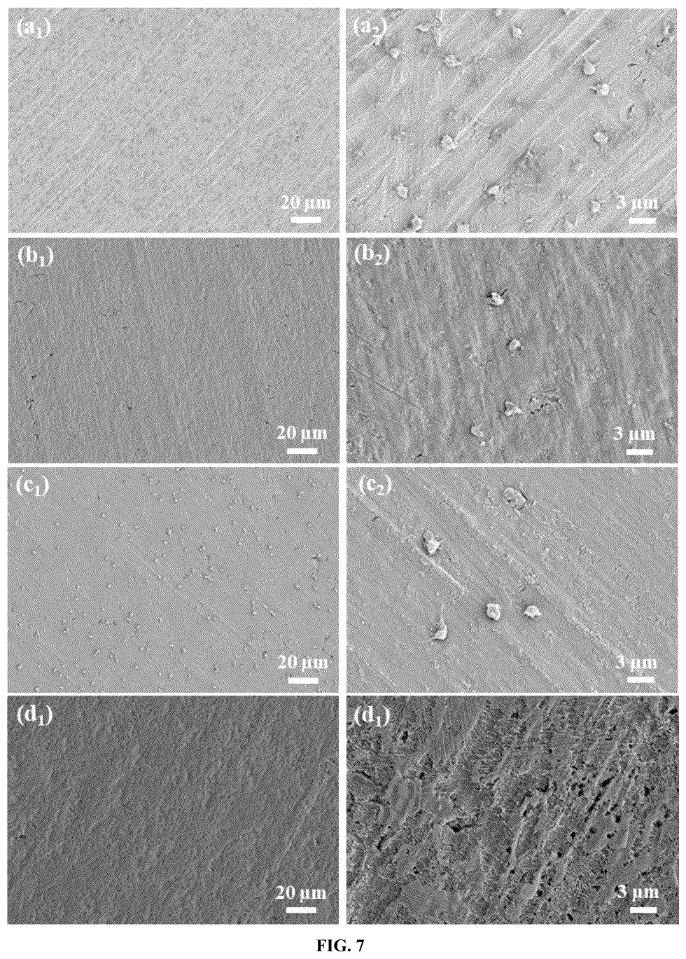

FIG. 7 showed SEM images of static adhesion with platelets on the surfaces of the titanium-based coating materials prepared in Example 5, Comparative Example 6, and Comparative Example 11, and the clean bare titanium foil prepared in Comparative Example 1; where (a₁) to (a₂) were Comparative Example 1, (b₁) to (b₂) were Comparative Example 6, (c₁) to (c₂) were Comparative Example 11, and (d₁) to (d₂) were Example 5. As shown in the FIG. 7, the surface of the superhydrophobic and self-cleaning anticoagulant composite coating material prepared in Example 5 had no platelet adhesion, which was superior to the anodized titanium dioxide coating material (Comparative Example 6) and the hydrophobic titanium-based coating material (Comparative Example 11), and was far superior to the bare titanium foil (Comparative Example 1). This showed that the superhydrophobic and self-cleaning anticoagulant composite coating material had an excellent static anticoagulant performance compared with the clean bare titanium foil, anodized titanium dioxide coating material and hydrophobic titanium-based coating material.

### (5.3) Dynamic Anticoagulant Performance—Dynamic Platelet Adhesion Test In Vitro

Test method: the superhydrophobic and self-cleaning anticoagulant composite coating material prepared in Example 5 and the clean bare titanium foil prepared in Comparative Example 1 were separately placed into a polymer tube partially filled with blood; the polymer tube formed a reclosable loop and rotated at 10 r/min to 40 r/min in a temperature-controlled environment to simulate arterial blood flow conditions. This was a Chandler circulatory system. After circulating for 3 h, the blood was emptied, and the samples were taken out and washed gently with a PBS solution 3 times until the unadhered platelets on the samples were washed away. After fixation, dehydration, critical point drying, and gold spraying, the adhesion of platelets on the surface of the samples was observed with a SEM.

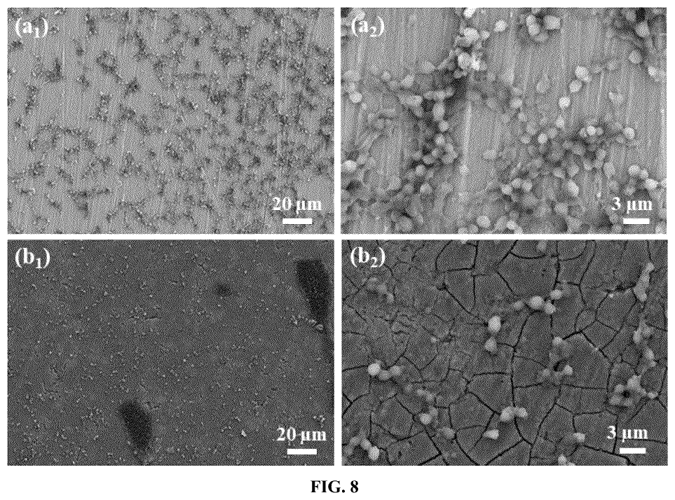

FIG. 8 showed SEM images of dynamic adhesion with platelets on the surfaces of the titanium-based coating material prepared in Example 5 and the clean bare titanium foil prepared in Comparative Example 1; where (a₁) to (a₂) were Comparative Example 1, and (b₁) to (b₂) were Example 5. It was seen from the figure that the surface of the superhydrophobic and self-cleaning anticoagulant composite coating material prepared in Example 5 had much less platelet adhesion than that of the clean bare titanium foil. This was because the superhydrophobic surface can trap air, forming an air layer on the surface of the material, thereby reducing the contact area with platelets (site adhesion). Therefore, even under dynamic conditions, the coating could effectively inhibit the adhesion of platelets.

In conclusion, the superhydrophobic and self-cleaning anticoagulant composite coating material has excellent hydrophobicity, desirable biocompatibility, self-cleaning performances of “anti-biofouling” for inhibiting bacterial adhesion, and excellent anticoagulant performances. The coating material can inhibit the adhesion of platelets, and can be applied as a surface coating of cardiovascular stents and implants, showing well self-cleaning and anticoagulant effects.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A superhydrophobic and self-cleaning anticoagulant composite coating material, comprising a titanium-based metal substrate, a titanium dioxide nanotube-based structure layer, and a hydrophobic modification layer that are sequentially laminated.

2. The superhydrophobic and self-cleaning anticoagulant composite coating material according to claim 1, wherein the hydrophobic modification layer is formed by a hydrophobic modifier; and the hydrophobic modifier comprises perfluorosilane and/or medium-chain and long-chain saturated fatty acids.

3. The superhydrophobic and self-cleaning anticoagulant composite coating material according to claim 2, wherein the perfluorosilane comprises perfluoroethoxysilane and/or perfluoromethoxysilane;
the perfluoroethoxysilane has a chemical formula of CF₃(CF₂)ₙCH₂CH₂Si(OC₂H₅)₃, and n is 5, 7, 9, or 11; and
the perfluoromethoxysilane has a chemical formula of CF₃(CF₂)ₘCH₂CH₂Si(OCH₃)₃, and m is 5 or 7.

4. The superhydrophobic and self-cleaning anticoagulant composite coating material according to claim 2, wherein the medium-chain and long-chain saturated fatty acids are C₁₂ saturated fatty acids to C₂₂ saturated fatty acids.

5. The superhydrophobic and self-cleaning anticoagulant composite coating material according to claim 4, wherein the C₁₂ saturated fatty acids to C₂₂ saturated fatty acids have a chemical formula of CH₃(CH₂)ₙCOOH, and a is 10 to 20.

6. A preparation method of a superhydrophobic and self-cleaning anticoagulant composite coating material comprising a titanium-based metal substrate, a titanium dioxide nanotube-based structure layer, and a hydrophobic modification layer that are sequentially laminated, the preparation method comprising the following steps:
placing the titanium-based metal substrate into an electrolyte, conducting anodization to form a titanium dioxide nanotube-based structure on a surface of the titanium-based metal substrate, to obtain an anodized titanium dioxide coating material; and
immersing the anodized titanium dioxide coating material into a solution of the hydrophobic modifier to form a hydrophobic modification layer, to obtain the superhydrophobic and self-cleaning anticoagulant composite coating material; wherein
the titanium-based metal substrate is selected from the group consisting of pure titanium, a titanium alloy sheet, and a titanium alloy foil; and
a cathode used in the anodization is prepared by a material selected from the group consisting of graphite, a platinum sheet, and a stainless steel.

7. The preparation method according to claim 6, wherein the electrolyte is selected from the group consisting of an aqueous inorganic salt alcoholic solution and an aqueous inorganic acid solution;
the aqueous inorganic salt alcoholic solution has 0.1 wt % to 0.5 wt % of an inorganic salt and 97.5 wt % to 97.9 wt % of an alcohol by concentration; and
the aqueous inorganic acid solution has a concentration of 0.1 wt % to 0.5 wt %.

8. The preparation method according to claim 7, wherein the inorganic salt in the aqueous inorganic salt alcoholic solution comprises a fluoride salt and/or a sulfate salt.

9. The preparation method according to claim 8, wherein the fluoride salt is at least one selected from the group consisting of NH₄F, NaF and KF.

10. The preparation method according to claim 8, wherein the sulfate salt comprises (NH₄)₂SO₄.

11. The preparation method according to claim 7, wherein the alcohol in the aqueous inorganic salt alcoholic solution is at least one selected from the group consisting of ethylene glycol, glycerol, and methanol.

12. The preparation method according to claim 7, wherein an inorganic acid in the aqueous inorganic acid solution comprises hydrofluoric acid.

13. (canceled)

14. The preparation method according to claim 6, wherein the titanium-based metal substrate is subjected to ultrasonic alcohol washing and ultrasonic water washing in sequence before use.

15. The preparation method according to claim 14, wherein the ultrasonic alcohol washing and the ultrasonic water washing each are conducted at 20° C. to 30° C. for 2 min to 10 min.

16. (canceled)

17. The preparation method according to claim 6, wherein during the anodization, an anode and the cathode have a spacing distance of 2 cm to 8 cm.

18. The preparation method according to claim 6, wherein the anodization is conducted at 0° C. to 30° C. with a voltage of 15 V to 55 V for 0.5 h to 5 h.

19. The preparation method according to claim 6, wherein the solution of the hydrophobic modifier has a concentration of 0.5 wt % to 10 wt %.

20. The preparation method according to claim 6, wherein the solution of the hydrophobic modifier has a solvent of alcohol.

21. The preparation method according to claim 6, wherein the immersing is conducted for 0.5 h to 24 h.

22. The preparation method according to claim 6, further comprising washing and drying an immersed material; wherein the drying is conducted at 80° C. to 140° C. for 0.5 h to 24 h.

23. (canceled)

24. (canceled)
